(19) United States
(12) Patent Application Publication
(10) Pub. No.: US 2024/0398608 A1
(43) Pub. Date: Dec. 5, 2024

(54) FLUID COLLECTION ASSEMBLIES INCLUDING AT LEAST ONE VERTICAL NONWOVEN MATERIAL

(71) Applicant: PUREWICK CORPORATION, Covington, GA (US)

(72) Inventors: Zhihui Yin, Lilburn, GA (US); Kamil Szymaniak, Dacula, GA (US); Kathleen Davis, Atlanta, GA (US); Michael Anderson, Monroe, GA (US); Brendan Tan-Fahed, Potomac, MD (US)

(21) Appl. No.: 18/689,532

(22) PCT Filed: Sep. 7, 2022
(86) PCT No.: PCT/US2022/042719

§ 371 Date: Mar. 6, 2024

Related U.S. Application Data
(60) Provisional application No. 63/241,575, filed on Sep. 8, 2021.

(51) Int. Cl. A61F 5/455; A61F 5/44

(52) U.S. Cl. CPC A61F 5/455; A61F 5/4401; A61F 5/4404

(57) ABSTRACT

An example fluid collection assembly includes a fluid impermeable layer that at least defines a chamber, at least one opening, and a fluid outlet. The fluid collection assembly also includes at least one porous material disposed in the chamber. The porous material includes at least one vertical nonwoven material that includes a plurality of fibers.

### CROSS-REFERENCE TO RELATED APPLICATION(S)

This application claims priority to U.S. Provisional Patent Application No. 63/241,575 filed on Sep. 8, 2021, the disclosure of which is incorporated herein, in its entirety, by this reference.

### BACKGROUND

A person or animal may have limited or impaired mobility so typical urination processes are challenging or impossible. For example, a person may experience or have a disability that impairs mobility. A person may have restricted travel conditions such as those experienced by pilots, drivers, and workers in hazardous areas. Additionally, sometimes bodily fluids collection is needed for monitoring purposes or clinical testing.

Urinary catheters, such as a Foley catheter, can address some of these circumstances, such as incontinence. Unfortunately, urinary catheters can be uncomfortable, painful, and can lead to complications, such as infections. Additionally, bed pans, which are receptacles used for the toileting of bedridden individuals are sometimes used. However, bedpans can be prone to discomfort, spills, and other hygiene issues.

### SUMMARY

Embodiments are directed to fluid collection assemblies including at least one vertical nonwoven material, fluid collection systems including the same, and methods of using and forming the same. In an embodiment, a fluid collection assembly is disclosed. The fluid collection assembly includes a fluid impermeable layer at least defining, a chamber, at least one opening, and a fluid outlet. The fluid collection assembly also includes at least one porous material disposed in the chamber. The at least one porous material includes at least one vertical nonwoven material. The vertical nonwoven material includes a plurality of fibers. The vertical nonwoven material is folded.

In an embodiment, a fluid collection system is disclosed. The fluid collection assembly includes a fluid collection assembly. The fluid collection assembly includes a fluid impermeable layer at least defining, a chamber, at least one opening, and a fluid outlet. The fluid collection assembly also includes at least one porous material disposed in the chamber. The at least one porous material includes at least one vertical nonwoven material. The vertical nonwoven material includes a plurality of fibers. The vertical nonwoven material is folded. The fluid collection system also includes a fluid storage container and a vacuum source. The chamber of the fluid collection assembly, the fluid storage container, and the vacuum source are in fluid communication with each that, when one or more bodily fluids are present in the chamber, a suction provided from the vacuum source to the chamber of the fluid collection assembly removes the one or more bodily fluids from the chamber and deposits the bodily fluids in the fluid storage container.

In an embodiment, a method of using a fluid collection assembly is disclosed. The method includes positioning at least one porous material extending across at least one opening defined by a fluid impermeable layer of the fluid collection assembly adjacent to a female urethral opening. The fluid impermeable layer at least defines a chamber and a fluid outlet. The at least one porous material is disposed in the chamber. The at least one porous material includes at least one vertical nonwoven material. The at least one vertical nonwoven material includes a plurality of fibers. The vertical nonwoven material is folded. The method also includes receiving one or more bodily fluids from the female urethral opening through the at least one opening and into the at least one porous material.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection assembly, according to an embodiment.

FIGS. 1B and 1C are cross-sectional schematics of the fluid collection assembly taken along planes 1B-1B and 1C-1C, respectively, shown in FIG. 1A.

FIG. 1D is a cross-sectional schematic of a portion of the fluid collection assembly taken from the box illustrated in FIG. 1C.

FIG. 2A is a cross-sectional schematic of a fluid collection assembly that includes a porous material having at least one additional material in addition to at least one vertical nonwoven material, according to an embodiment.

FIG. 2B is a cross-sectional schematic of the fluid collection assembly taken along plane 2B-2B shown in FIG. 2A.

FIG. 3 is a cross-sectional schematic of a fluid collection assembly, according to an embodiment.

FIG. 4 is a block diagram of a fluid collection system for fluid collection, according to an embodiment.

### DETAILED DESCRIPTION

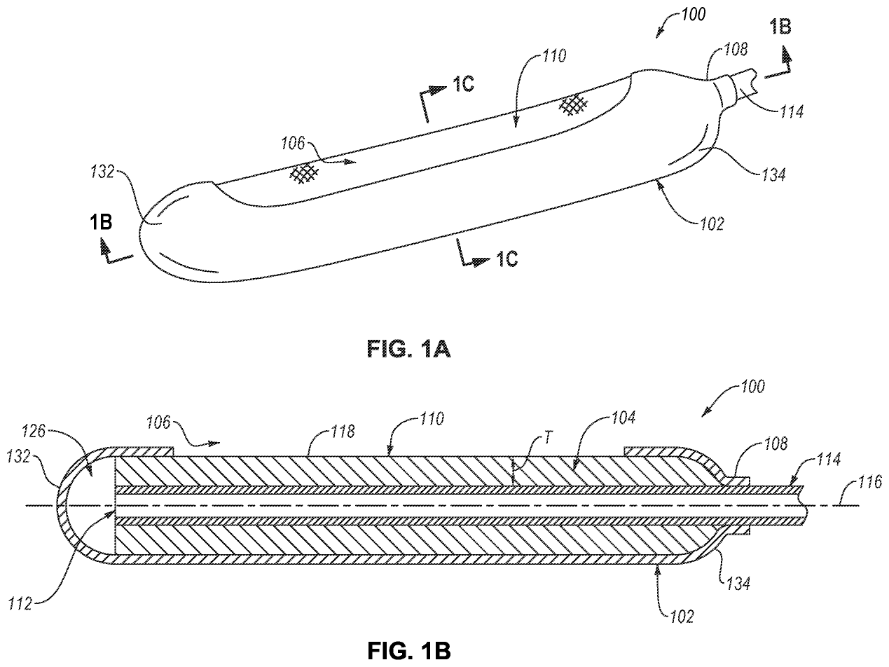

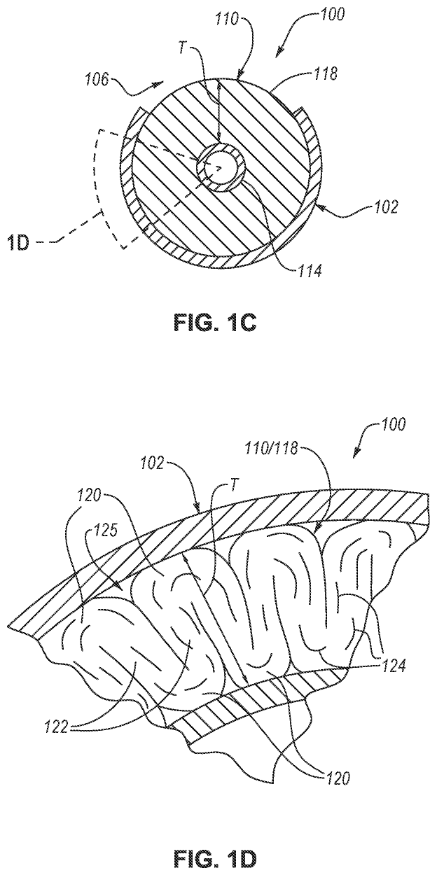

Embodiments are directed to fluid collection assemblies including at least one vertical nonwoven material, fluid collection systems including the same, and methods of using and forming the same. An example fluid collection assembly includes a fluid impermeable layer (e.g., a fluid impermeable barrier) that at least defines a chamber, at least one opening, and a fluid outlet. The fluid collection assembly also includes at least one porous material disposed in the chamber. The porous material includes at least one vertical nonwoven material that includes a plurality of fibers. A vertical nonwoven material, for example, includes a nonwoven web that is folded vertically, as discussed in more detail with regards to FIG. 1D.

During use, the fluid collection assembly may be positioned on an individual such that the opening is positioned adjacent to a female urethral opening or receives a male urethral opening (i.e., on a penis). The individual may discharge one or more bodily fluids, such as urine, blood, or sweat. The bodily fluids may flow into the chamber and be received into the porous material. The bodily fluids may be removed from the chamber via the fluid outlet. In an embodiment, a suction may be applied to the chamber from a vacuum source which removes the bodily fluids from the chamber.

Some conventional fluid collection assemblies include a porous material other than a vertical nonwoven material. The porous materials of such conventional fluid collection assemblies may include a porous thin film, a gauze disposed on a polyethylene terephthalate or spun nylon fiber core, or a cover sheet disposed on a cross-lapped nonwoven filtration material. However, it has been found that the porous materials used in such conventional fluid collection assemblies may not be able to quickly capture bodily fluids and transport bodily fluids. As used herein, “capture bodily fluids” and the like refers to the ability of a porous material to receive bodily fluids and “transport bodily fluids” and the like refers to the ability of the porous material to quickly move the bodily fluids towards an outlet (e.g., the fluid outlet or an inlet of a conduit). The porous materials used in conventional fluid collection assemblies that are unable to efficiently capture bodily fluids may result in leakage of the bodily fluids, especially when the individual using the conventional fluid collection assemblies discharges a large quantity of bodily fluids over a short period of time (e.g., urinates). The porous materials used in conventional fluid collection assemblies that are unable to quickly transport bodily fluids are unable to quickly dry which, unless such conventional fluid collection assemblies are changed relatively frequently (e.g., changed after at least most 12 hours of use, at most 18 hours or use, or, with significant risk of skin degradation, at most 24 hours of use), results in skin degradation. Further, porous materials used in conventional fluid collection assemblies that are unable to quickly transport bodily fluids may become saturated with bodily fluids which prevents the porous materials from receiving more bodily fluids which, in turn, may result in leakage of the bodily fluids.

The fluid collection assemblies disclosed herein are an improvement over such conventional fluid collection assemblies at least because the fluid collection assemblies include a porous material that includes at least one vertical nonwoven material. It has been surprisingly found that the vertical nonwoven material is able to quickly capture bodily fluids and transport bodily fluids. As such, the vertical nonwoven material may better prevent leakage of the bodily fluids and better maintain the porous material dry than if the porous material included other porous materials that are commonly used in conventional fluid collection assemblies. For example, the fluid collection assemblies disclosed herein may be used for prolonged periods of time substantially without causing skin degradation because the fluid collection assemblies disclosed herein include the vertical nonwoven material. The prolonged period of time that the fluid collection assemblies disclosed herein may be used include period of times of 24 hours or more, about 30 hours or more, about 36 hours or more, about 42 hours or more, about 48 hours or more, or in ranges of about 24 hours to about 36 hours, about 30 hours to about 42 hours, or about 36 hours to about 48 hours

FIG. 1A is an isometric view of a fluid collection assembly 100, according to an embodiment. FIGS. 1B and 1C are cross-sectional schematics of the fluid collection assembly 100 taken along planes 1B-1B and 1C-1C, respectively, shown in FIG. 1A. The fluid collection assembly is an example of a fluid collection assembly configured to receive bodily fluids from a female urethral opening. The fluid collection assembly 100 includes a fluid impermeable layer 102. The fluid impermeable layer 102 at least defines a chamber 104, at least one opening 106, and a fluid outlet 108. The fluid collection assembly 100 also includes at least one porous material 110 disposed in the chamber 104 that extends across the opening 106. The porous material 110 includes at least one vertical nonwoven material.

The fluid impermeable layer 102 at least partially defines a chamber 104 (e.g., interior region) and an opening 106. The fluid impermeable layer 102 temporarily stores the bodily fluids in the chamber 104. The fluid impermeable layer 102 may be formed of any suitable fluid impermeable material(s), such as a fluid impermeable polymer (e.g., silicone, polypropylene, polyethylene, polyethylene terephthalate, neoprene, a polycarbonate, etc.), a metal film, natural rubber, another suitable material, any other fluid impermeable material disclosed herein, or combinations thereof. As such, the fluid impermeable layer 102 substantially prevents the bodily fluids from passing through the fluid impermeable layer 102. In an example, the fluid impermeable layer 102 may be air permeable and fluid impermeable. In such an example, the fluid impermeable layer 102 may be formed of a hydrophobic material that defines a plurality of pores. At least one or more portions of at least an outer surface of the fluid impermeable layer 102 may be formed from a soft and/or smooth material, thereby reducing chaffing.

The opening 106 provides an ingress route for bodily fluids to enter the chamber 104. The opening 106 may be defined by the fluid impermeable layer 102 such as by an inner edge of the fluid impermeable layer 102. For example, the opening 106 is formed in and extends through the fluid impermeable layer 102 thereby enabling bodily fluids to enter the chamber 104 from outside of the fluid collection assembly 100.

In some examples, the fluid impermeable layer 102 may define a fluid outlet 108 sized to receive the conduit 114. The at least one conduit 114 may be disposed in the chamber 104 via the fluid outlet 108. The fluid outlet 108 may be sized and shaped to form an at least substantially fluid tight seal against the conduit 114 or the at least one tube thereby substantially preventing the bodily fluids from escaping the chamber 104.

As previously discussed, the fluid collection assembly 100 includes porous material 110 disposed in the chamber 104. The porous material 110 may cover at least a portion (e.g., all) of the opening 106. The porous material 110 may include a vertical nonwoven material.

The vertical nonwoven material may be formed from any suitable vertical nonwoven material. In an embodiment, the vertical nonwoven material may be formed from synthetic fibers. Examples of synthetic fibers includes polyester, polypropylene, or nylon. In an embodiment, the vertical nonwoven material may be formed from natural fibers which may be more sustainable and biodegradable than the synthetic fibers. Examples of natural fibers includes cellulose, cotton, and bamboo. In an embodiment, the vertical nonwoven material may be formed from natural and synthetic fibers.

In an embodiment, the vertical nonwoven material may be configured to wick and/or otherwise allow transport of any bodily fluids away from the opening 106, thereby preventing the bodily fluids from escaping the chamber 104. The permeable properties referred to herein may be wicking, capillary action, diffusion, or other similar properties or processes, and are referred to herein as “permeable” and/or “wicking.” Such “wicking” and/or “permeable” properties may not include absorption of the bodily fluids into at least a portion of the vertical nonwoven material. Put another way, substantially no absorption or solubility of the bodily fluids into the vertical nonwoven material may take place after the vertical nonwoven material is exposed to the bodily fluids and removed from the bodily fluids for a time. While no absorption or solubility is desired, the term “substantially no absorption” may allow for nominal amounts of absorption and/or solubility of the bodily fluids into the vertical nonwoven material (e.g., absorbency), such as less than about 30 wt % of the dry weight of the vertical nonwoven material, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the vertical nonwoven material. In an embodiment, the vertical nonwoven material may include at least one absorbent or adsorbent material.

In an embodiment, the vertical nonwoven material may by hydrophilic. The hydrophilicity of the vertical nonwoven material may cause the vertical nonwoven material to quickly capture bodily fluids therein thereby preventing or at least inhibiting leakage of bodily fluids caused by a large discharge of bodily fluids over a short period of time. The vertical nonwoven material may be hydrophilic when the vertical nonwoven material exhibits a contact angle with water (a major constituent of bodily fluids) that is about 0° to about 10°, about 5° to about 15°, about 10° to about 20°, about 15° to about 25°, about 20° to about 30°, about 25° to about 35°, about 30° to about 40°, about 35° to about 45°, about 40° to about 50°, about 45° to about 55°, about 50° to about 60°, about 55° to about 65°, about 60° to about 70°, about 65° to about 75°, about 70° to about 80°, about 75° to about 85°, or about 80° to 90°. Generally, increasing the hydrophilicity of the vertical nonwoven material (i.e., decreasing the contact angle between the vertical nonwoven material and water) increases the quantity of bodily fluids that the vertical nonwoven material may receive over a certain period of time. However, increasing the hydrophilicity of the vertical nonwoven material may increase the quantity of bodily fluids that are retained in the vertical nonwoven material after the vertical nonwoven material receives the bodily fluids. As such, the hydrophilicity of the vertical nonwoven material may be selected based on balancing the need to receive bodily fluids quickly while also keeping the porous material 110 dry. For example, a fluid collection assembly 100 configured to be used with an individual with a large bladder for short periods of time may include a vertical nonwoven material exhibiting a hydrophilicity that is greater than a vertical nonwoven material of a fluid collection assembly 100 configured to be used with an individual with an average to small sized bladder for long period of time. It is noted that porous materials of at least some conventional fluid collection assemblies are selected to be hydrophobic to improve the fluid transport thereof. However, it was unexpectedly found that vertical nonwoven materials exhibit quick fluid transport even when the vertical nonwoven materials are hydrophilic.

In an embodiment, the hydrophilicity of the vertical nonwoven material may be an inherent property of the material(s) (e.g., fibers) used to form the vertical nonwoven material. In an embodiment, the hydrophilicity of the material(s) vertical nonwoven material may be changed by at least one of impurities or functional groups added to the vertical nonwoven material, otherwise treating the vertical nonwoven material, or coating the vertical nonwoven material with a material that exhibits a hydrophilicity that is different than the vertical nonwoven material.

In an embodiment, the vertical nonwoven material may be hydrophobic. The vertical nonwoven material may be hydrophobic when the vertical nonwoven material exhibits a contact angle with water that is about 90° to about 120°, about 105° to about 135°, about 120° to about 150°, about 135° to about 165°, or greater than 150°. The hydrophobic vertical nonwoven material may more quickly transport the bodily fluids received thereby than if the vertical nonwoven material is hydrophilic.

The vertical nonwoven material of the porous material 110 may be selected to exhibit a density of about 5 kg/m²/cm to about 10 kg/m²/cm, about 7.5 kg/m²/cm to about 12.5 kg/m²/cm, about 10 kg/m²/cm to about 15 kg/m²/cm, about 12.5 kg/m²/cm to about 17.5 kg/m²/cm, about 15 kg/m²/cm to about 20 kg/m²/cm, about 17.5 kg/m²/cm to about 22.5 kg/m²/cm, about 20 kg/m²/cm to about 25 kg/m²/cm, about 22.5 kg/m²/cm to about 27.5 kg/m²/cm, about 25 kg/m²/cm to about 30 kg/m²/cm, about 27.5 kg/m²/cm to about 32.5 kg/m²/cm, about 30 kg/m²/cm to about 35 kg/m²/cm, about 32.5 kg/m²/cm to about 37.5 kg/m²/cm, about 35 kg/m²/cm to about 37.5 kg/m²/cm, about 35 kg/m²/cm to about 40 kg/m²/cm, about 37.5 kg/m²/cm to about 42.5 kg/m²/cm, about 40 kg/m²/cm to about 45 kg/m²/cm, about 42.5 kg/m²/cm to about 47.5 kg/m²/cm, or about 45 kg/m²/cm to about 50 kg/m²/cm. Generally, increasing the density of the vertical nonwoven material increases the strength of the vertical nonwoven material. However, increasing the density of the vertical nonwoven material may decrease the porosity of the vertical nonwoven material which decreases the quantity of bodily fluids that may be temporarily stored in the porous material 110 and decreases the flow rate of the bodily fluids through the vertical nonwoven material. As such, the density of the vertical nonwoven material may be selected based on balancing the desired strength, porosity, and flow rate of the bodily fluids through the vertical nonwoven material.

The vertical nonwoven material of the porous material 100 may be selected to exhibit a thickness T that is greater than about 1 mm, such as in ranges of 1 mm to about 3 mm, about 2 mm to about 4 mm, about 3 mm to about 5 mm, about 4 mm to about 6 mm, about 5 mm to about 7 mm, about 6 mm to about 8 mm, about 7 mm to about 9 mm, about 8 mm to about 10 mm, about 9 mm to about 11 mm, about 10 mm to about 12 mm, about 11 mm to about 13 mm, about 12 mm to about 14 mm, about 13 mm to about 15 mm, about 14 mm to about 16 mm, about 15 mm to about 18 mm, about 17 mm to about 20 mm, about 19 mm to about 22 mm, about 21 mm to about 25 mm, or about 24 mm to about 30 mm. Increasing the thickness T of the vertical nonwoven material generally increases the volume of bodily fluids that may be temporarily stored in the vertical nonwoven material, and allows greater flexibility in selecting the density and basis weight of the vertical nonwoven material. However, the thickness T of the nonwoven material may be limited by the size and functionality of the fluid collection assembly 100. For example, the thickness T of the vertical nonwoven material must be selected such that the vertical nonwoven material may be disposed in the chamber 104, along with any other items that may also be disposed in the chamber 104, such as at least one of at least one additional material of the porous material 110, or a conduit 110. Further, increasing the thickness T of the vertical nonwoven material may make removing substantially all of the bodily fluids from the chamber 104 difficult since the increased thickness T dilutes the vacuum pressure in the porous material 110.

The vertical nonwoven material of the porous material 110 may be selected to exhibit a basis weight of about 1 gm/m² to about 25 g/m², about 10 g/m² to about 75 g/m², about 50 g/m² to about 100 g/m², about 75 g/m² to about 125 g/m², about 100 g/m² to about 150 g/m², about 125 g/m² to about 175 g/m², about 150 g/m² to about 200 g/m², about 175 g/m² to about 225 g/m², about 200 g/m² to about 250 g/m², about 225 g/m² to about 275 g/m², about 250 g/m² to about 300 g/m², about 275 g/m² to about 325 g/m², about 300 g/m² to about 375 g/m², about 350 g/m² to about 450 g/m², about 400 g/m² to about 500 g/m², about 450 g/m² to about 550 g/m² or about 500 g/m² to about 600 g/m². The basis weight of the vertical nonwoven material is a function of the density and thickness of the vertical nonwoven material. As such, the basis weight of the vertical nonwoven material may be selected for any of the same reasons as the density and thickness of the vertical nonwoven material.

As previously discussed, the vertical nonwoven material is formed from a plurality of fibers 124. The plurality of fibers 124 may exhibit an average length and an average lateral dimension (e.g., diameter). In an example, the plurality of fibers 124 may be selected to exhibit an average length that is about 500 μm to about 2 mm, about 1 mm to about 3 mm, about 2 mm to about 4 mm, about 3 mm to about 5 mm, about 4 mm to about 6 mm, about 5 mm to about 7 mm, about 6 mm to about 8 mm, about 7 mm to about 9 mm, about 8 mm to about 1 cm, about 9 mm to about 1.2 cm, about 1 cm to about 1.4 cm, about 1.2 cm to about 1.6 cm, about 1.4 cm to about 1.8 cm, about 1.6 cm to about 2 cm, about 1.8 cm to about 2.25 cm, about 2 cm to about 2.5 cm, about 2.25 cm to about 2.75 cm, about 2.5 cm to about 3 cm, about 2.75 cm to about 3.25 cm, about 3 cm to about 3.5 cm, about 3.25 cm to about 3.75 cm, about 3.5 cm to about 4 cm, about 3.75 cm to about 4.25 cm, about 4 cm to about 4.5 cm, about 4.25 cm to about 4.75 cm, about 4.5 cm to about 5 cm, about 4.75 cm to about 5.5 cm, about 5 cm to about 6 cm, about 5.5 cm to about 6.5 cm, about 6 cm to about 7 cm, about 6.5 cm to about 7.5 cm, about 7 cm to about 8 cm, about 7.5 cm to about 8.5 cm, about 8 cm to about 9 cm, about 8.5 cm to about 9.5 cm, or about 9 cm to about 10 cm. In an example, the fibers 124 may exhibit an average lateral dimension that is about 1 μm to about 2 μm, about 1.5 μm to about 3 μm, about 2 μm to about 4 μm, about 3 μm to about 5 μm, about 4 μm to about 7 μm, about 6 μm to about 10 μm, about 8 μm to about 12.5 μm, about 10 μm to about 15 μm, about 12.5 μm to about 17.5 μm, about 15 μm to about 20 μm, about 17.5 μm to about 25 μm, about 20 μm to about 30 μm, about 25 μm to about 35 μm, about 30 μm to about 40 μm, about 35 μm to about 45 μm, about 40 μm to about 50 μm, about 45 μm to about 55 μm, about 50 μm to about 60 μm, about 55 μm to about 65 μm, about 60 μm to about 70 μm, about 65 μm to about 75 μm, about 70 μm to about 80 μm, about 75 μm to about 85 μm, about 80 μm to about 90 μm, about 85 μm to about 95 μm, or about 90 μm to about 100 μm. The average length and average lateral dimension of the fibers 124 may be selected such that the fibers 124 exhibits an average aspect ratio. For example, the average length and average lateral dimension of the fibers 124 may be selected such that the fibers 124 exhibit an average aspect ratio (average length:average lateral dimension) of about 25:1 to about 75:1, about 50:1 to about 100:1, about 75:1 to about 150:1, about 100:1 to about 200:1, about 150:1 to about 250:1, about 200:1 to about 300:1, about 250:1 to about 350:1, about 300:1 to about 400:1, about 350:1 to about 450:1, about 400:1 to about 500:1, about 450:1 to about 550:1, about 500:1 to about 600:1, about 550:1 to about 650:1, about 600:1 to about 700:1, about 650:1 to about 750:1, about 700:1 to about 800:1, about 750:1 to about 850:1, about 800:1 to about 900:1, about 850:1 to about 950:1, or about 900:1 to about 1,000:1.

The average length, average lateral dimension, and the average aspect ratio of the fibers 124 may be selected based on a number of factors. In an example, increasing the aspect ratio (e.g., increasing average length) of the fibers 124 may increase the mechanical binding of the fibers 124. For instance, increasing the aspect ratio of the fibers 124 facilitates entanglement of the fibers 124 which increases the strength and durability of the vertical nonwoven material. The entanglement of the fibers 124 may also preclude or minimize the amount of other binding techniques that are applied to the vertical nonwoven material, such as heat, chemical binding, or other mechanical binding (e.g., further entanglement caused by needle punching or high pressure water jets). However, increasing the aspect ratio of the fibers 124 may make dispersion of the fibers 124 more difficult (e.g., uniformity of the vertical nonwoven material difficult). Further, increasing the aspect ratio may limit the type of nonwoven webs that may form the vertical nonwoven material. For instance, fibers 124 with large average lengths (e.g., large aspect ratios) may not be used in carded webs and may have to be used in air laid webs. In an example, decreasing the aspect ratio may decrease the entanglement of the fibers 124 thereby necessitating further binding of the fibers 124. As such, the average length, average lateral dimension, and average aspect ratio of the fibers 124 may be selected based on the desired strength, mechanical binding between the fibers, the amount of processing of the vertical nonwoven material (e.g., is further processing to increasing the binding via heat, etc. desired), the type of nonwoven web 118 that includes the fibers 124, the uniformity of the fibers 124, etc.

Generally, the average person discharges urine at a rate of about 6 ml/s to about 50 ml/s, such as at a rate of about 10 ml/s to about 25 ml/s. The rate at which the person urinate may vary, such as based on the size of the person and the age of the person. The vertical nonwoven material may be selected to capture and transport the bodily fluids at a rate that is comparable to the rate at which the individual discharged bodily fluids to prevent leaks. For example, the vertical nonwoven material may be selected to capture and transport the bodily fluids at a rate that is greater than about 6 ml/s, greater than about 10 ml/s, greater than about 20 ml/s, greater than about 30 ml/s, greater than about 40 ml/s, greater than about 50 ml/s, or in ranges of about 6 ml/s to about 10 ml/s, about 8 ml/s to about 12 ml/s, about 10 ml/s to about 15 ml/s, about 12.5 ml/s to about 17.5 ml/s, about 15 ml/s to about 20 ml/s, about 17.5 ml/s to about 22.5 ml/s, about 20 ml/s to about 25 ml/s, about 22.5 ml/s to about 27.5 ml/s, about 25 ml/s to about 30 ml/s, about 27.5 ml/s to about 35 ml/s, about 30 ml/s to about 40 ml/s, about 35 ml/s to about 45 ml/s, or about 40 ml/s to about 50 ml/s.

The rate at which the vertical nonwoven material captures and transports the bodily fluids may depend on a number of factors. In an example, the rate at which the vertical nonwoven material captures and transports the bodily fluids may depend inversely on the density and weight basis of the vertical nonwoven material, wherein increasing the density and/or weight basis of the vertical nonwoven material may decrease the rate at which the vertical nonwoven material captures and transports the bodily fluids and vice versa. In an example, the rate at which the vertical nonwoven material captures and transports the bodily fluids may depend on the material (e.g., hydrophilicity of the material) that forms the fibers 124. In an example, the rate at which the vertical nonwoven material captures and transports the bodily fluids may increase with increasing thickness T since increasing the thickness T increases the cross-sectional area through which the bodily fluids may flow. In an example, the rate at which the vertical nonwoven material captures and transports the bodily fluids may depend on the type of nonwoven web (e.g., carded web, needle punched web, etc.) since each type of nonwoven web may exhibit different rate at which the vertical nonwoven material captures and transports the bodily fluids.

FIG. 1D is a cross-sectional schematic of a portion of the fluid collection assembly 100 taken from the box illustrated in FIG. 1C. The vertical nonwoven material is formed from a nonwoven web 118 that is folded. The folded nonwoven web 118 may include a plurality of folded portions 120 and a plurality of intermediate portions 122 extending between the folded portions 120. The folded nonwoven web 118 may include an outer surface adjacent to the fluid impermeable layer 102 and an opposing inner surface (e.g., defining a bore that received the conduit 114). The folder portion 120 may extend generally parallel to the outer and inner surfaces of folded nonwoven web 118. The intermediate portions 122 may extend between the outer and inner surfaces of the folded nonwoven web 118. In an embodiment, the folded nonwoven web 118 may be positioned in the chamber 104 such that the folded portions 120 extend generally parallel to a longitudinal (e.g., central) axis 116 of the fluid collection assembly 100 (e.g., generally parallel to a longitudinal axis of the porous material 110) and/or extend circumferentially when the porous material 110 exhibits a generally cylindrical shape. The folded nonwoven web 118 may be positioned in the chamber 104 such that the intermediate portions 122 extend generally parallel to the longitudinal axis 116 of the fluid collection assembly 100 (e.g., generally parallel to a longitudinal axis of the porous material 110) and/or extend radially when the porous material 110 exhibits a generally cylindrical shape.

As previously discussed, the vertical nonwoven material includes a plurality of fibers 124. In an embodiment, the nonwoven web 118 that forms the vertical nonwoven material may include a plurality of generally oriented fibers 124. The generally oriented fibers 124 may improve the ability of the vertical nonwoven material to capture bodily fluids and transport bodily fluids. The generally oriented fibers 124 may also improve the mechanical properties of the vertical nonwoven material. As used herein, the fibers 124 are “generally aligned” when a certain percentage of the fibers 124 are substantially parallel to each other. The certain percentage of the fibers 124 refers to at least about 70% of the fibers 124, more preferably at least about 80% of the fibers 124, more preferable 90% of the fibers 124, and even more preferably at least about 95% of the fibers 124. The fibers 124 are generally parallel to each other when the certain percentage of fibers 124 are parallel to each other ±30° more preferable ±20°, more preferably ±10°, or even more preferably ±5°.

The nonwoven material 118 may be disposed in the chamber 104 such that the fibers 124 of the folded portions 120 are generally oriented circumferentially and the fibers 124 of the intermediate portions 122 are generally oriented radially. Not wishing to be bound to theory, the circumferentially orientation of the fibers 124 of the folded portions 120 may cause the bodily fluids received by the vertical nonwoven material to initially preferentially disperse circumferentially and the radial orientation of the fibers 124 of the intermediate portions 124 may cause the bodily fluids to initially preferentially disperse radially into the porous material 110. Causing the bodily fluids to initially disperse circumferentially and radially quickly disperses the bodily fluids throughout a large volume of the vertical nonwoven material thereby allowing the vertical nonwoven material to quickly capture and transport the bodily fluids. It is noted that the fibers 124 do not inhibit flow of the bodily fluids in a direction that is generally parallel to the longitudinal axis 116, especially after the fibers 124 are wetted. Further, dispersing the bodily fluids throughout the vertical nonwoven material increases the surface area of any bodily fluids that may remain in the vertical nonwoven material after removing the bodily fluids from the porous material 110. The large surface area facilitates evaporation of the remaining bodily fluids with the air flow through the porous material 110. In an embodiment, the fibers 124 are randomly oriented or may be oriented differently than what is shown in FIG. 1D.

In an embodiment, as illustrated, folding the nonwoven web 118 may cause the formation of gaps 125 that extending generally parallel to the longitudinal axis 116. The gaps 125 may facilitate fluid flow in a direction that is generally parallel to the longitudinal axis 116. However, the nonwoven web 118 may be folded or compressed by the fluid impermeable layer 102 to minimize the size of the gaps 125 to prevent pooling of the bodily fluids in the chamber 104. For example, the nonwoven web 118 may be folded or compressed by the fluid impermeable layer 102 to cause the gaps 125 to exhibit a dimension measured perpendicular to the longitudinal axis 116 that is less than about 1 mm, less than about 0.75 mm, less than about 0.5 mm, or less than about 0.25 mm.

As previously discussed, the vertical nonwoven material may be formed from at least one nonwoven web 118 that is folded. The vertical nonwoven material may be formed from any suitable nonwoven web. In an embodiment, the nonwoven web includes at least one carded web. The carded web includes a plurality of fibers 124 that may be generally oriented in the same direction. The generally same orientation of the fibers 124 of the carded web cause the carded web to be anisotropic. For example, the strength of the carded web is greatest when a force applied thereto is generally parallel to the fibers 124 but the strength of the carded web decreases as the force applied thereto becomes more oblique or perpendicular to the orientation of the fibers 124. As such, the carded web may need to be positioned in the chamber 104 to mitigate forces being applied to the carded web that are not generally parallel to the orientation of the fibers 124 or requires addition binding between the fibers 124 (e.g., heat or chemical) to prevent unsatisfactory wear of the carded web. The initial flow the bodily fluids through the carded web may vary depending on whether the bodily fluids are flowing parallel, obliquely, or perpendicular to the orientation of the fibers 124. As such, selecting the nonwoven web 118 to include the carded web allows for selecting the strength and flow characteristics of the porous material 110 based on the orientation of the fibers 124. Even though the fibers 124 are generally oriented, the orientation of each of the fibers 124 may slightly vary which causes the porosity of the carded web to be sufficiently high that the carded web exhibits any of the density, thickness, basis weight, and flow rates disclosed herein.

In an embodiment, the nonwoven web 118 may include at least one needle punched web. The needle punched web may be formed from a sheet including a plurality of fibers 124. The sheet may include a plurality of randomly oriented fibers 124 (e.g., the fibers 124 are generally parallel to and randomly oriented in the plane), or generally oriented fibers 124 (e.g., a carded web) since the orientation of the fibers 124 may better facilitate flow of the bodily fluids therethrough. A plurality of needles (e.g., a plurality of barbed needles) are inserted into the sheet in a direction that is generally parallel to a thickness of the sheet which causes some of the fibers 124 to become entangled and interlocked. For example, insertion of the needles into the sheet cause some of the fibers 124 to reorient and migrate from the surface of the sheet to an interior thereof to form columns. The entanglement of the fibers 124 caused by the insertion of the needles may sufficiently entangle the fibers 124 such that no additional binding is necessary to bond the fibers 124 together. The entanglement of the fibers 124 may cause the needle punched web to exhibit more isotropic properties compared to the carded web and, thus, may not require specific orientation in the chamber 104 or additional binding of the fibers 124. The needle punched web may exhibit good flow features. For example, the needles extending into the sheet may form divots which facilitate flow of the bodily fluids vertically through the needle punched web.

In an embodiment, the nonwoven web 118 may include at least one air laid web. The air laid web may exhibit a plurality of randomly oriented fibers 124. The plurality of random fibers 124 may exhibit a length that is sufficiently large that the fibers 124 become entangled and do not need be bounded together or the fibers 124 may be bonded. Due to the random orientation of the fibers 124, the air laid web tends to be isotropic and exhibit a high porosity. Similar, due to the random orientation of the fibers 124, the air laid web may exhibit a high loft. The air laid web may be formed from fibers 124 that cannot be carded (e.g., short fibers).

In an embodiment, the nonwoven web 118 may include at least one spunlaced web. The spunlaced web is formed by providing a sheet that includes randomly oriented fibers 124 or a carded web. High pressure water jets that are generally parallel to the thickness of the sheet are directed towards the sheet. Similar to the needle punched web, the high pressure jets of water cause some of the fibers 124 to migrate from an exterior of the sheet to an interior thereof to form columns. Thus, the spunlaced web may function similar to the needle punched web, namely that the spunlaced web may be more isotropic than the carded web and includes divots. However, the spunlaced web may exhibit at least one of a density that is less than, a thickness that is greater than, or a base weight that is less than the needle punched web. As such, the spunlaced web may be more delicate (e.g., less durable or softer) than the needle punched web. The more delicate spunlaced web may more comfortably contact the skin of the patient than the needle punched web.

It is currently believed that the carded web, needle punched web, the air laid web, and the spunlaced web are the preferred nonwoven webs to be included in the vertical nonwoven material. However, it is noted that the vertical nonwoven material may include one or more nonwoven webs other than the carded web, needle punched web, the air laid web, and the spunlaced web. In an example, the vertical nonwoven material may include a wet laid web even though the wet laid web may exhibit low durability compared to the other nonwoven webs disclosed herein. In an example, the vertical nonwoven material may include spunbond or meltblown nonwoven webs even though such nonwoven webs may exhibit too low of porosity for some applications.

In an embodiment, the nonwoven web does not include a horizontal or cross lapped nonwoven material. The horizontal or cross lapped nonwoven material include nonwoven webs that are folded differently than the vertical nonwoven materials discussed above. It has been found that the different folds of the horizontal and cross lapped nonwoven materials causes the horizontal and cross lapped nonwoven materials to capture and/or transport bodily fluids significantly less quickly than a comparable (e.g., exhibit same material, hydrophilicity, basis weight, density, etc.) vertical nonwoven material. In an embodiment, the nonwoven web includes a horizontal or cross lapped nonwoven material.

The folded nonwoven web 118 may be formed from a sheet. When resting the sheet on a horizontal planar surface, the folded portions 120 may extend parallel to the horizontal planar surface and the intermediate portions 122 may extend vertically from the horizontal planar surface. The folded nonwoven web 118 may then be rolled to form the cylindrical folded nonwoven web 118 illustrated in FIG. 1C.

Referring back to FIGS. 1A-1C, in an embodiment, as illustrated, the porous material 110 only or substantially only includes the vertical nonwoven material. In such an embodiment, the vertical nonwoven material may define a bore that is configured to receive the conduit 114 and the vertical nonwoven material extends from the bore to the fluid impermeable layer 102. When the porous material 110 includes only or substantially only the vertical nonwoven material, all of the porous material 110 is able to quickly capture and transport the bodily fluids. However, it is noted that the porous material 110 may include at least one additional material even though such additional material may decrease at least one of the ability of the porous material 110 to capture and/or transport the bodily fluids.

The porous material 110 may at least substantially completely fill the portions of the chamber 104 that are not occupied by the conduit 114. In some examples, the porous material 110 may not substantially completely fill the portions of the chamber 104 that are not occupied by the conduit 114. In such an example, the fluid collection assembly 100 includes the reservoir 126 disposed in the chamber 104.

The reservoir 126 is a substantially unoccupied portion of the chamber 104. The reservoir 126 may be defined between the fluid impermeable layer 102 and porous material 110. The bodily fluids that are in the chamber 104 may flow through the porous material 110 to the reservoir 126. The reservoir 126 may retain of the bodily fluids therein.

The bodily fluids that are in the chamber 104 may flow through the porous material 110 to the reservoir 126. The fluid impermeable layer 102 may retain the bodily fluids in the reservoir 126. While depicted in the distal end region 132, the reservoir 126 may be located in any portion of the chamber 104 such as the proximal end region 134. The reservoir 126 may be located in a portion of the chamber 104 that is designed to be located in a gravimetrically low point of the fluid collection assembly when the fluid collection assembly is worn.

In some examples (not shown), the fluid collection assembly 100 may include multiple reservoirs, such as a first reservoir that is located at the portion of the chamber 104 closest to the inlet of the conduit 114 (e.g., distal end region 132) and a second reservoir that is located at the portion of the of the chamber 104 that is at or near proximal end region 134). In another example, the porous material 110 is spaced from at least a portion of the conduit 114, and the reservoir 126 may be the space between the porous material 110 and the conduit 114.

The conduit 114 may be at least partially disposed in the chamber 104. The conduit 114 may be used to remove the bodily fluids from the chamber 104. The conduit 114 includes at least one wall defining an inlet 112, an outlet (not shown) downstream from the inlet 112, and a passageway. The outlet of the conduit 114 may be operably coupled to a vacuum source, such as a vacuum pump for withdrawing fluid from the chamber 104 through the conduit 114. For example, the conduit 114 may extend into the fluid impermeable layer 102 from the proximal end region 134 and may extend to the distal end region 132 to a point proximate to the reservoir 126 therein such that the inlet 112 is in fluid communication with the reservoir 126. The conduit 114 fluidly couples the chamber 104 with the fluid storage container (not shown) or the vacuum source (not shown).

The conduit 114 may extend through a bore in the porous material 110. In an embodiment, the conduit 114 extends from the fluid outlet 108, through the bore, to a location that is proximate to the reservoir 126. In such an embodiment, the inlet 112 may not extend into the reservoir 126 and, instead, the inlet 112 may be disposed within the porous material 110 or at a terminal end thereof. For example, an end of the conduit 114 may be coextensive with or recessed within the porous material 110. In an embodiment, the conduit 114 is at least partially disposed in the reservoir 126 and the inlet 112 may be extended into or be positioned in the reservoir 126. In an embodiment, the inlet 112 may be positioned aft of the reservoir 126. The bodily fluids collected in the fluid collection assembly 100 may be removed from the chamber 104 via the conduit 114.

Locating the inlet 112 at or near a location expected to be the gravimetrically low point of the chamber 104 when worn by an individual enables the conduit 114 to receive more of the bodily fluids than if inlet 112 was located elsewhere and reduce the likelihood of pooling (e.g., pooling of the bodily fluids may cause microbe growth and foul odors). For instance, the bodily fluids in the porous material 110 may flow in any direction due to capillary forces. However, the bodily fluids may exhibit a preference to flow in the direction of gravity, especially when at least a portion of the porous material 110 is saturated with the bodily fluids. Accordingly, one or more of the inlet 112 or the reservoir 126 may be located in the fluid collection assembly 100 in a position expected to be the gravimetrically low point in the fluid collection assembly 100 when worn by an individual, such as the distal end region 132.

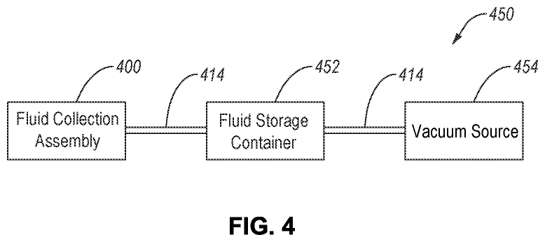

The inlet 112 and the outlet of the conduit 114 are configured to fluidly couple (e.g., directly or indirectly) the vacuum source (not shown) to the chamber 104 (e.g., the reservoir 126). As the vacuum source (FIG. 4) applies a vacuum/suction in the conduit 114, the bodily fluids in the chamber 104 (e.g., at the distal end region 132 such as in the reservoir 126) may be drawn into the inlet 112 and out of the fluid collection assembly 100 via the conduit 114. In some examples, the conduit 114 may be frosted or opaque (e.g., black) to obscure visibility of the bodily fluids therein.

As previously discussed, the conduit 114 may be configured to be at least insertable into the chamber 104. In an example, the conduit 114 may be positioned in the chamber 104 such that a terminal end of the conduit 114 is spaced from the fluid impermeable layer 102 or other components of the fluid collection assembly 100 that may at least partially obstruct or block the inlet 112. Further, the inlet 112 of the conduit 114 may be offset relative to a terminal end of the porous material 110 such that the inlet 112 is closer to the proximal end region 134 of the fluid collection assembly 100 than the terminal end of the porous material 110. Offsetting the inlet 112 in such a manner relative to the terminal end of the porous material 110 allows the inlet 112 to receive bodily fluids directly from the porous material 110 and, due to hydrogen bonding, pulls more bodily fluids from the porous material 110 into the conduit 114.

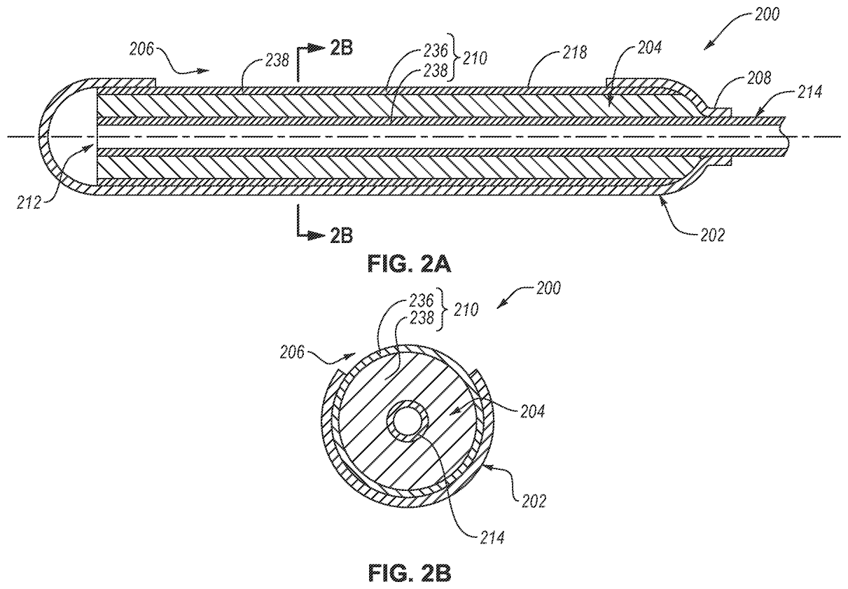

As previously discussed, the porous materials of the fluid collection assemblies disclosed herein may include at least one additional material (e.g., a fluid permeable membrane) in addition to the vertical nonwoven material. For example, FIG. 2A is a cross-sectional schematic of a fluid collection assembly 200 that includes a porous material 210 having at least one additional material in addition to at least one vertical nonwoven material 238, according to an embodiment. FIG. 2B is a cross-sectional schematic of the fluid collection assembly 200 taken along plane 2B-2B shown in FIG. 2A. Except as otherwise disclosed herein, the fluid collection assembly 200 is the same as or substantially similar to any of the fluid collection assemblies disclosed herein. For example, the fluid collection assembly 200 may include a fluid impermeable layer 202 at least defining a chamber 204, at least one opening 206, and a fluid outlet 208. Also, the vertical nonwoven material 238 of the porous material 210 may be the same or substantially similar to any of the vertical nonwoven materials disclosed herein.

The additional material 236 may include any suitable porous material, such as a porous sheet. In an example, the additional material 236 may include gauze (e.g., a silk, linen, or cotton gauze), another soft fabric, another smooth fabric, a horizontal lapped nonwoven material, a cross lapped nonwoven material, a porous polymer (e.g., nylon, polyester, polyurethane, polyethylene, polypropylene, etc.) structure or an open cell foam (e.g., spun nylon fiber), or any other suitable porous material. In an example, the additional material 236 may include a hydrophobic material (e.g., a material exhibiting a contact angle with water that is greater than) 90°. In an example, the additional material 236 may include a vertical nonwoven material exhibiting a density, basis weight, thickness, different average fiber length, different average fiber lateral dimension, different average fiber aspect ratio, or different rate at which the vertical nonwoven material captures and transports the bodily fluids than what is disclosed above.

In an embodiment, as illustrated, the additional material 236 is disposed on an outer surface of the vertical nonwoven material 238 (e.g., between the fluid impermeable layer 202 and the vertical nonwoven material 238) such that the additional material 236 extends across the opening 206 and contacts the individual during use (i.e., the vertical nonwoven material 238 may form a fluid permeable support for the additional material 236). The additional material 236 may be disposed on the vertical nonwoven material 238 to make the fluid collection assembly 200 more comfortable to use and/or improve capture of the bodily fluids. In an example, an individual may find direct contact between the vertical nonwoven material 238 and the sensitive vaginal region of the individual uncomfortable, for instance, due to the surface roughness or fibers protruding from the vertical nonwoven material 238. In such an example, the additional material 236 may include a material (e.g. gauze) that is smoother or otherwise more comfortable than the vertical nonwoven material 238. In an example, as previously discussed, the hydrophilicity of the vertical nonwoven material 238 may be limited to facilitate removing bodily fluids therefrom. However, limiting the hydrophilicity of the vertical nonwoven material 238 may limit the ability of the vertical nonwoven material 238 to capture bodily fluids. As such, the additional material 236 may be selected to exhibit a hydrophilicity that is greater than (i.e., a contact angle with water that is less than) the vertical nonwoven material 238 which allows the additional material 236 to capture bodily fluids more quickly than the vertical nonwoven material 238. When the additional material 236 exhibits a hydrophilicity that is greater than the vertical nonwoven material 238, the additional material 236 may exhibit a thickness that is significantly less than the thickness of the vertical nonwoven material. The smaller thickness of the additional material 236 decreases the volume of bodily fluids that are retained in the additional material 236 that have to be evaporated by air flow through the chamber 204.

In an embodiment, the additional material 236 may be positioned between the vertical nonwoven material 238 and the conduit 214 instead of or in addition to being disposed on the outer surface of the vertical nonwoven material 238.

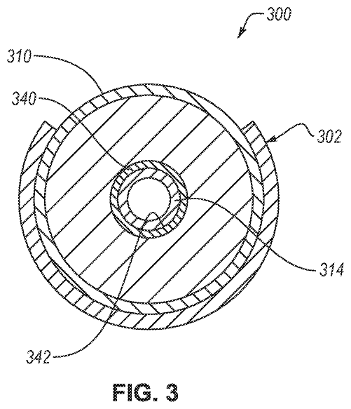

In some embodiments, the fluid collection assemblies disclosed herein may include an element configured to allow the shape of the fluid collection assemblies to be controllably changed and/or maintain a selected shape. FIG. 3 is a cross-sectional schematic of a fluid collection assembly 300, according to an embodiment. Except as otherwise disclosed herein, the fluid collection assembly 300 is the same as or substantially similar to any of the fluid collection assemblies disclosed herein. For example, the fluid collection assembly 300 may include a fluid impermeable layer 302, a porous material 310, and a conduit 314. The porous material 310 may include any of the vertical nonwoven materials disclosed herein.

The fluid collection assembly 300 includes at least one shape memory material 340 configured to prevent or at least inhibit bodily fluids leaking from the fluid collection assembly 300. For example, bodily fluids may leak from the fluid collection assembly because, initially, the fluid collection assembly 300 may exhibit a poor fit with the region about the urethral opening. The poor fit may cause gaps to be present between the porous material 310 and the region about the urethral opening. These gaps may provide locations through which the bodily fluids may flow without being received by the porous material and/or locations at which bodily fluids may leave the porous material 310. To minimize formation of the gaps, the fluid collection assembly 300 includes the shape memory material 340. The shape memory material 340 is configured to be manipulated (e.g., bent or otherwise shaped) which, in turn, causes the fluid collection assembly 300 to exhibit a shape that matches the anatomical shape of the patient and to conform to the shape of the vaginal region. In other words, the shape memory material 340 allows the fluid collection assembly 300 to exhibit a good fit with the region about the urethral opening.

The shape memory material 340 may be sized, shaped, and positioned in the fluid collection assembly 300 to cause at least a portion of the fluid collection assembly 300 to retain a selected shape (e.g., geometric configuration). In an embodiment, the shape memory material 340 is configured to be bent, shaped, or otherwise deformed (hereafter collectively referred to as “shape,” “shaped,” or “shaping”). In an example, the shape memory material 340 is configured to be shaped along an entire length thereof. Allowing the shape memory material 340 to be shaped along the entire length thereof may allow the fluid collection assembly 300 to exhibit a shape that substantially corresponds to the anatomical features of the patient. For example, the shape memory material 300 may exhibit a first (e.g., initial) shape. The fluid collection assembly 300 may exhibit the first configuration (i.e., a generally linear shape shape) when the shape memory material 340 exhibits the first shape (e.g., a generally straight cylindrical shape). The shape memory material 340 may be shaped to exhibit a second shape that is different than the first shape. The fluid collection assembly 300 may exhibit the second configuration (e.g., a generally curved cylindrical shape) when the shape memory material 340 exhibits the second shape. The second configuration of the fluid collection assembly 300 may better correspond to the shape of the region about the urethral opening than the first configuration.

The shape memory material 340 may include a shape memory polymer or a metal (e.g., shape memory metal). Generally, the shape memory material 340 is composed to adopt an intermediate or permanent shape in response to a stimuli. For example, the shape memory material 340 may exhibit a first (e.g., initial) shape and may be switched from the first shape to a second shape by the stimuli, wherein the second shape is different than the first shape. The shape memory material 340 may also be switched from the second shape back to the first shape or a third shape that is different than the first and second shapes in response to the stimuli.

The stimuli may include an external physical force (e.g., bending force), heat, electrical bias, or a magnetic field. While the term “shape memory” is used to describe some of the “shape memory materials” herein, it should be understood that, in some examples, the material modified by the term “shape memory” may not necessarily need to return to a preselected shape upon application of a stimuli, as understood as the classical definition of the “shape memory material.” Rather, at least some of the shape memory materials disclosed herein may simply hold a selected shape when bent, set, or cured into a specific shape and/or when cooled in a specific shape, regardless of the stimuli applied thereto after. The shape memory materials may be returned to the original shape or changed to a new shape by application of stimuli. For example, a metal foil bent to a first shape may be utilized as the shape memory material 340, whereinafter the metal foil may be modified to a second shape via physical force applied thereto or via heating. However, in some embodiments, the shape memory material 340 may exhibit a selected shape, as discussed above and application of the stimuli may cause the shape memory material to deform (e.g., elastically deform or bend) into an intermediate shape. In such embodiments, the shape memory material 340 may return to the first initial shape upon removal of the stimuli such that the shape memory material 340 does not maintain the intermediate shape.

In an embodiment, the shape memory material 340 may include metal, such as an elemental metal, an alloy, or shape memory alloy. Suitable shape memory metals may include aluminum, silver, copper, iron, nickel, zinc, tin, beryllium, or the like. Suitable shape memory alloys may include standard steels, stainless steel, carbon alloy steel, head treated steel, galvanized steel, aluminum alloys, nickel-titanium alloys (e.g., Nitinol, Ni—Ti—Cu, Ni—Ti, Co, or the like), copper-based alloys (e.g., Cu—Zn—Al, Cu—Al—Ni, Cu—Al—Sn, or the like), Co—Cr—Ni—Mo alloys (e.g., Elgiloy® or the like), or any other alloy having shape memory characteristics. As explained above, the shape memory metals or alloys may merely be metals or alloys that may be shaped to a selected configuration. In some examples, the shape memory metals or alloys may return to a primary shape when an external stimuli is applied thereto. In some examples, the outer surface of the shape memory metal may be coated with a polymer, anodized, passivated, or otherwise treated to prevent corrosion.

Shape memory polymers (“SMPs”) may include polyurethane-based SMPs such as a copolymer (e.g., copolyester, polyurethane, polyetherester, etc.) including blocks of one or more of poly (ε-caprolactone), polyethyleneterephthalate (PET), polyethyleneoxide (PEO), glycol polyethylene (PEG), polystyrene, polymethylmethacrylate (PMMA), Polybutylmethacrylate (PBMA), poly(N,N-butadiene), poly(N-methyl-N-oxazoline), polytetrahydrofuran, or poly(butylene terephthalate); thermoplastic polymers such as polyether ether ketone (PEEK), nylon, acetal, polytetrafluoroethylene (PTFE), polypropylene, polyethylene, acrylonitrile butadiene styrene (ABS), polysulphone, or the like; Polynorbonene; other deformable polymers; or any other shape memory polymer.

In an embodiment, the fluid collection assembly 300 includes the shape memory material 340 disposed on an exterior surface 342 of the conduit 314. The shape memory material 340 is distinct from the conduit 314. The shape memory material 340 may be a coating, foil, or other structure disposed on an exterior surface 342 of the conduit 314. In an example, the shape memory material 340 is disposed around the entire circumference of the exterior surface 342 of the conduit 314 which may increase the ability of the shape memory material to be manipulated into a selected shape and maintain the selected shape. In an example, the shape memory material 340 is disposed along substantially all of the length of the conduit 314 that is disposed in the chamber 304. In such an example, the shape memory material 340 may affect the shape of the fluid collection assembly 300 globally. In an example, the shape memory material 340 is only disposed on a portion of the exterior surface 342 of the conduit 314.

Further examples of shape memory material and other elements configured to allow the shape of the fluid collection assemblies to be controllably changed and/or maintain a selected shape are disclosed in International Application No. PCT/US2020/042262 filed Jul. 16, 2020, U.S. Patent Application Publication No. 2022/0117774 filed Oct. 19, 2021, and U.S. Patent Application Publication No. 2022/0151817 filed Nov. 16, 2021, the disclosure of each of which is incorporated herein, in its entirety, by this reference.

The fluid collection assemblies shown in FIGS. 1A-3 are examples of female fluid collection assemblies that are configured to collect bodily fluids from females (e.g., collect urine from a female urethral opening). However, the porous materials and shape memory materials disclosed herein may be used in a male fluid collection assemblies. Examples of male fluid collection assemblies that may include the porous materials and shape memory materials discussed herein are disclosed in PCT Patent Application No. PCT/US2021/039866 filed on Jun. 30, 2021 and U.S. patent application Ser. No. 16/433,773 filed on Jun. 6, 2019, the disclosure of each of which is incorporated herein, in its entirety, by this reference.

FIG. 4 is a block diagram of a fluid collection system 450 for fluid collection, according to an embodiment. The fluid collection system 450 includes a fluid collection assembly 400, a fluid storage container 452, and a vacuum source 454. The fluid collection assembly 400 may be the same or substantially similar to any of the fluid collection assemblies disclosed herein. The fluid collection assembly 400, the fluid storage container 452, and the vacuum source 454 may be fluidly coupled to each other via one or more conduits 414. For example, fluid collection assembly 400 may be operably coupled to one or more of the fluid storage container 452 or the vacuum source 454 via the conduit 414. The bodily fluids collected in the fluid collection assembly 400 may be removed from the fluid collection assembly 400 via the conduit 414 which protrudes into the fluid collection assembly 400. For example, an inlet of the conduit 414 may extend into the fluid collection assembly 400, such as to a reservoir therein. The outlet of the conduit 414 may extend into the fluid collection assembly 400 or the vacuum source 454. Suction force may be introduced into the chamber of the fluid collection assembly 400 via the inlet of the conduit 414 responsive to suction (e.g., vacuum) force applied at the outlet of the conduit 414.

The suction force may be applied to the outlet of the conduit 414 by the vacuum source 454 either directly or indirectly. The suction force may be applied indirectly via the fluid storage container 452. For example, the outlet of the conduit 414 may be disposed within the fluid storage container 452 and an additional conduit 414 may extend from the fluid storage container 452 to the vacuum source 454. Accordingly, the vacuum source 454 may apply suction to the fluid collection assembly 400 via the fluid storage container 452. The suction force may be applied directly via the vacuum source 454. For example, the outlet of the conduit 414 may be disposed within the vacuum source 454. An additional conduit 414 may extend from the vacuum source 454 to a point outside of the fluid collection assembly 400, such as to the fluid storage container 452. In such examples, the vacuum source 454 may be disposed between the fluid collection assembly 400 and the fluid storage container 452.

The fluid storage container 452 is sized and shaped to retain bodily fluids therein. The fluid storage container 452 may include a bag (e.g., drainage bag), a bottle or cup (e.g., collection jar), or any other enclosed container for storing bodily fluids such as urine. In some examples, the conduit 414 may extend from the fluid collection assembly 400 and attach to the fluid storage container 452 at a first point therein. An additional conduit 414 may attach to the fluid storage container 452 at a second point thereon and may extend and attach to the vacuum source 454. Accordingly, a vacuum (e.g., suction) may be drawn through fluid collection assembly 400 via the fluid storage container 452. Bodily fluids, such as urine, may be drained from the fluid collection assembly 400 using the vacuum source 454.

The vacuum source 454 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 454 may provide a vacuum or suction to remove bodily fluids from the fluid collection assembly 400. In some examples, the vacuum source 454 may be powered by one or more of a power cord (e.g., connected to a power socket), one or more batteries, or even manual power (e.g., a hand operated vacuum pump). In some examples, the vacuum source 454 may be sized and shaped to fit outside of, on, or within the fluid collection assembly 400. For example, the vacuum source 454 may include one or more miniaturized pumps or one or more micro pumps. The vacuum sources 454 disclosed herein may include one or more of a switch, a button, a plug, a remote, or any other device suitable to activate the vacuum source 454.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. A fluid collection assembly, comprising:
a fluid impermeable layer at least defining:
a chamber;
at least one opening; and
a fluid outlet;
at least one porous material disposed in the chamber, the at least one porous material including at least one vertical nonwoven material, the at least one vertical nonwoven material including a plurality of fibers that are generally aligned, wherein the vertical nonwoven material is folded.

2. The fluid collection assembly of claim 1, wherein the at least one vertical nonwoven material defines a bore configured to receive a conduit.

3. The fluid collection assembly of claim 2, further comprising a shape memory foil or coating disposed on at least a portion of a surface of the conduit.

4. The fluid collection assembly of claim 1, wherein the at least one vertical nonwoven material is hydrophilic.

5. The fluid collection assembly of claim 1, wherein the at least one vertical nonwoven material includes at least one of polyester, polypropylene, nylon, cellulose, or cotton.

6. The fluid collection assembly of claim 1, wherein the at least one vertical nonwoven material includes bamboo.

7. The fluid collection assembly of claim 1, wherein the at least one vertical nonwoven material exhibits a thickness of about 1.5 mm to about 20 mm.

8. (canceled)

9. The fluid collection assembly of claim 1, wherein the at least one vertical nonwoven material exhibits a surface density of about 140 g/m² to about 250 g/m².

10. The fluid collection assembly of claim 1, wherein the at least one vertical nonwoven material exhibits a density of about 60 kg/m³ to about 250 kg/m³.

11. (canceled)

12. The fluid collection assembly of claim 1, wherein the at least one vertical nonwoven material includes a plurality of folded portions and a plurality of intermediate portions extending between the folded portions.

13. The fluid collection assembly of claim 12, wherein the plurality of fibers of each of the plurality of the folded portions of the at least one vertical nonwoven material are generally aligned circumferentially and the plurality of fibers of each of the plurality of intermediate portions of the at least one vertical nonwoven material are generally aligned radially.

14. (canceled)

15. The fluid collection assembly of claim 1, wherein the at least one vertical nonwoven material includes a single nonwoven material that extends from a conduit disposed in the chamber to the fluid impermeable layer.

16. The fluid collection assembly of claim 1, wherein the at least one porous material further includes at least one additional material disposed on the at least one vertical nonwoven material.

17. The fluid collection assembly of claim 16, wherein the at least one additional material includes at least one gauze.

18. The fluid collection assembly of claim 1, wherein the at least one vertical nonwoven material extends across the at least one opening.

19. The fluid collection assembly of claim 1, further comprising a conduit at least partially disposed in the chamber and at least one shape memory material disposed on at least a portion of an exterior surface of the conduit.

20. A fluid collection system, comprising:
a fluid collection assembly including:
a fluid impermeable layer at least defining:
a chamber;
at least one opening; and
a fluid outlet;
at least one porous material disposed in the chamber, the at least one porous material including at least one vertical nonwoven material, the at least one vertical nonwoven material including a plurality of fibers that are generally aligned, wherein the vertical nonwoven material is folded;
a fluid storage container; and
a vacuum source;
wherein the chamber of the fluid collection assembly, the fluid storage container, and the vacuum source are in fluid communication with each other so that, when one or more bodily fluids are present in the chamber, a suction provided from the vacuum source to the chamber of the fluid collection assembly removes the one or more bodily fluids from the chamber and deposits the bodily fluids in the fluid storage container.

21. A method of using a fluid collection assembly, the method comprising:
positioning at least one porous material extending across at least one opening defined by a fluid impermeable layer of the fluid collection assembly adjacent to a female urethral opening, the fluid impermeable layer at least defining a chamber and a fluid outlet, the at least one porous material disposed in the chamber, the at least one porous material including at least one vertical nonwoven material, the at least one vertical nonwoven material including a plurality of fibers, wherein the vertical nonwoven material is folded; and
receiving one or more bodily fluids from the female urethral opening through the at least one opening and into the at least one porous material.

22. The method of claim 21, further comprising removing the fluid collection assembly from the female urethral opening 24 hours or more after receiving one or more bodily fluids from the female urethral opening.

23. The fluid collection assembly of claim 1, wherein at least about 80% of the plurality of fibers are parallel to each other within about ±20°.
